(19) United States
(12) Patent Application Publication
(10) Pub. No.: US 2010/0249604 A1
(43) Pub. Date: Sep. 30, 2010

(54) SYSTEMS AND METHODS FOR MAKING AND USING A MOTOR DISTALLY-POSITIONED WITHIN A CATHETER OF AN INTRAVASCULAR ULTRASOUND IMAGING SYSTEM

(75) Inventors: Roger N. Hastings, Maple Grove, MN (US); Michael J. Pikus, Golden Valley, MN (US); Tat-Jin Teo, Sunnyvale, CA (US); Leonard B. Richardson, Brooklyn Park, MN (US); Kevin D. Edmunds, Ham Lake, MN (US)

Correspondence Address: Boston Scientific Corporation;c/o Frommer Lawrence & Haug LLP, 745 Fifth Avenue, New York, NY, 10151, US

(73) Assignee: Boston Scientific Corporation, Valencia, CA (US)

(21) Appl. No.: 12/415,791

(22) Filed: Mar. 31, 2009

(51) Int. Cl. A61B 8/14

(52) U.S. Cl. 600/467

(57) ABSTRACT

An imaging core, that is configured and arranged for insertion into a catheter, includes a mirror disposed at a distal end of a rotatable driveshaft; a motor coupled to the driveshaft and including a rotatable magnet and at least two magnetic field windings disposed around at least a portion of the magnet on a rigid slotted material; and at least one fixed transducer positioned between the motor and the rotatable mirror. The driveshaft extends through an aperture in the magnet to allow passage of the driveshaft through the at least one transducer to the rotatable mirror. At least one transducer conductor is electrically coupled to the at least one transducer and in electrical communication with the proximal end of the catheter. At least one motor conductor is electrically coupled to the magnetic field windings and in electrical communication with the proximal end of the catheter.

### TECHNICAL FIELD

The present invention is directed to the area of intravascular ultrasound imaging systems and methods of making and using the systems. The present invention is also directed to intravascular ultrasound systems that include imaging cores distally positioned within catheters, the imaging cores including motors for rotating the imaging cores, as well as methods of making and using the imaging cores, motors, and intravascular ultrasound systems.

### BACKGROUND

Intravascular ultrasound (“IVUS”) imaging systems have proven diagnostic capabilities for a variety of diseases and disorders. For example, IVUS imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4 is a schematic longitudinal cross-sectional view of one embodiment of an imaging core disposed in a distal end of a lumen of a catheter, the imaging core including a motor, one or more stationary transducers, and a rotating mirror, according to the invention;

FIG. 5 is a schematic perspective view of one embodiment of a rotating magnet and associated windings, according to the invention;

FIG. 6 is a schematic top view of one embodiment of windings disposed on a thin film, according to the invention;

FIG. 7 is a schematic perspective view of one embodiment of a three-phase winding geometry configured and arranged for forming a rotating magnetic field around a motor, according to the invention;

FIG. 8 is a schematic side view of one embodiment of a portion of a transducer coupled to a portion of a slotted magnetic field winding, transducer conductors coupled to the transducer extend through one of the slots of the magnetic field winding, according to the invention; and

FIG. 9 is a schematic transverse cross-sectional view of one embodiment of a transducer, according to the invention;

### DETAILED DESCRIPTION

The present invention is directed to the area of intravascular ultrasound imaging systems and methods of making and using the systems. The present invention is also directed to intravascular ultrasound systems that include imaging cores distally positioned within catheters, the imaging cores including motors for rotating the imaging cores, as well as methods of making and using the imaging cores, motors, and intravascular ultrasound systems.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; and 6,945,938; as well as U.S. Patent Application Publication Nos. 20060253028; 20070016054; 20070038111; 20060173350; and 20060100522, all of which are incorporated by reference.

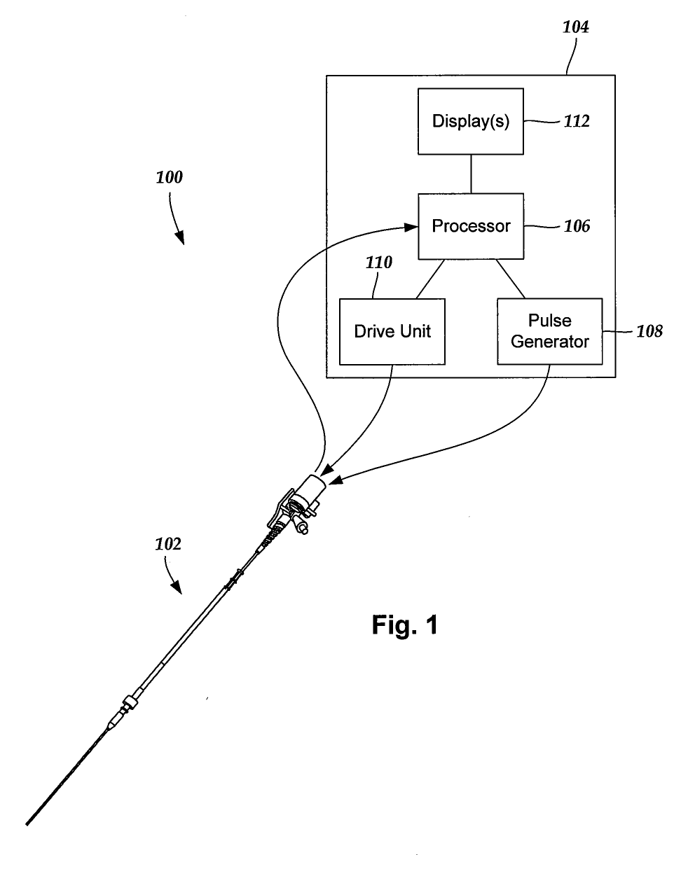

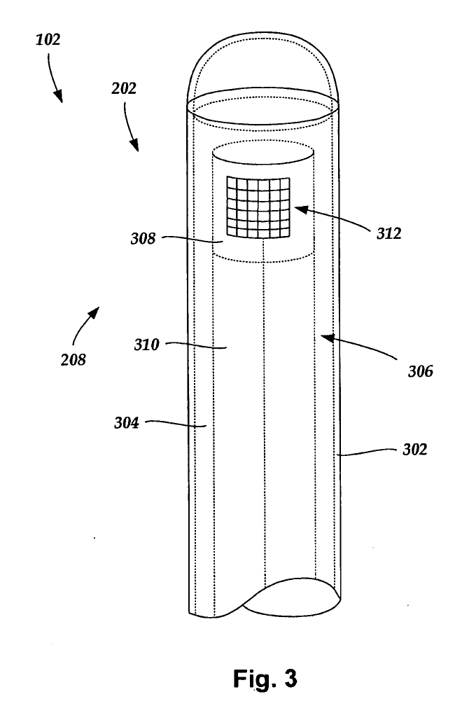

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric pulses that may be input to one or more transducers (312 in FIG. 3) disposed in the catheter 102. In at least some embodiments, mechanical energy from a pullback motor disposed within the drive unit 110 may be used to provide translational movement of an imaging core (306 in FIG. 3) disposed in the catheter 102.

In at least some embodiments, electric pulses transmitted from the one or more transducers (312 in FIG. 3) may be input to the processor 106 for processing. In at least some embodiments, the processed electric pulses from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical pulses transmitted from the pulse generator 108, the rotation rate of the imaging core (306 in FIG. 3) by the drive unit 110, the velocity or length of the pullback of the imaging core (306 in FIG. 3) by the drive unit 110, or one or more properties of one or more images formed on the one or more displays 112.

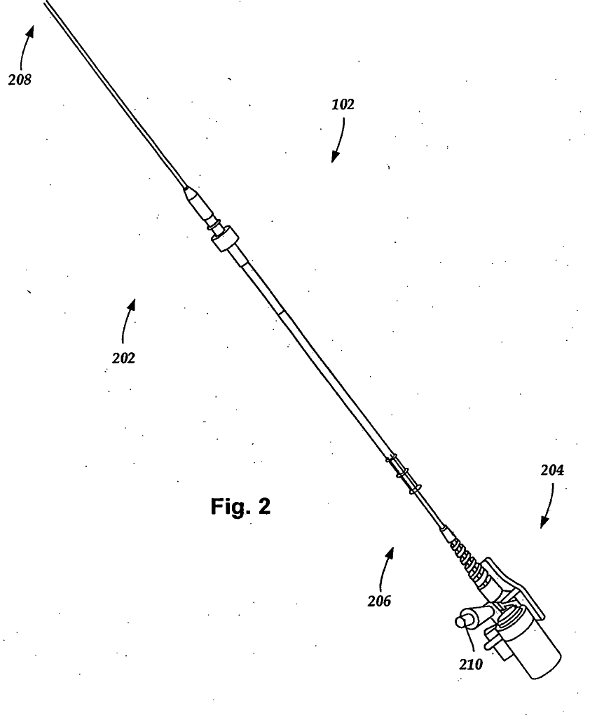

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device 308 coupled to a distal end of a rotatable driveshaft 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device 308 and employed to transmit and receive acoustic pulses. In a preferred embodiment (as shown in FIG. 3), an array of transducers 312 are mounted to the imaging device 308. In other embodiments, a single transducer may be employed. In yet other embodiments, multiple transducers in an irregular-array may be employed. Any number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

The one or more transducers 312 may be formed from one or more known materials capable of transforming applied electrical pulses to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like.

The pressure distortions on the surface of the one or more transducers 312 form acoustic pulses of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic pulses.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

In at least some embodiments, the imaging core 306 may be rotated about a longitudinal axis of the catheter 102. As the imaging core 306 rotates, the one or more transducers 312 emit acoustic pulses in different radial directions. When an emitted acoustic pulse with sufficient energy encounters one or more medium boundaries, such as one or more tissue boundaries, a portion of the emitted acoustic pulse is reflected back to the emitting transducer as an echo pulse. Each echo pulse that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic pulses transmitted and the echo pulses received.

As the one or more transducers 312 rotate about the longitudinal axis of the catheter 102 emitting acoustic pulses, a plurality of images are formed that collectively form a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays (112 in FIG. 1).

In at least some embodiments, the drive unit (110 in FIG. 1) is used to provide translational movement to the imaging core 306 within the lumen of the catheter 102 while the catheter 102 remains stationary. For example, the imaging core 306 may be advanced (moved towards the distal end of the catheter 102) or retracted/pulled back (moved towards the proximal end of the catheter 102) within the lumen 304 of the catheter 102 while the catheter 102 remains in a fixed location within patient vasculature (e.g., blood vessels, the heart, and the like). During longitudinal movement (e.g., pullback) of the imaging core 306, an imaging procedure may be performed, wherein a plurality of cross-sectional images are formed along a longitudinal length of patient vasculature.

In at least some embodiments, the pullback distance of the imaging core is at least 5 cm. In at least some embodiments, the pullback distance of the imaging core is at least 10 cm. In at least some embodiments, the pullback distance of the imaging core is at least 15 cm. In at least some embodiments, the pullback distance of the imaging core is at least 20 cm. In at least some embodiments, the pullback distance of the imaging core is at least 25 cm.

The quality of an image produced at different depths from the one or more transducers 312 may be affected by one or more factors including, for example, bandwidth, transducer focus, beam pattern, as well as the frequency of the acoustic pulse. The frequency of the acoustic pulse output from the one or more transducers 312 may also affect the penetration depth of the acoustic pulse output from the one or more transducers 312. In general, as the frequency of an acoustic pulse is lowered, the depth of the penetration of the acoustic pulse within patient tissue increases. In at least some embodiments, the IVUS imaging system 100 operates within a frequency range of 5 MHz to 60 MHz.

In at least some embodiments, the catheter 102 with one or more transducers 312 mounted to the distal end 208 of the imaging core 306 may be inserted percutaneously into a patient via an accessible blood vessel, such as the femoral artery, at a site remote from the selected portion of the selected region, such as a blood vessel, to be imaged. The catheter 102 may then be advanced through the blood vessels of the patient to the selected imaging site, such as a portion of a selected blood vessel.

It is desirable to have uniform rotation of the imaging core 306 during operation. When the catheter 102 is advanced through blood vessels of the patient, the catheter 102 may navigate one or more tortuous regions or one or more narrow regions which may press against one or more portions of the catheter 102 and cause a non-uniform rotation (e.g., a wobble, a vibration, or the like) of the imaging core 306 during operation. Non-uniform rotation may lead to the distortion of a subsequently-generated IVUS image. For example, the subsequently-generated IVUS image may be blurred.

In conventional systems, a rotational motor is disposed in a proximal portion of the catheter 302 or in a unit to which the proximal portion of the catheter is attached. Due to the distance between a proximally-positioned rotational motor and an imaging core and the tortuous nature of the vasculature into which the distal end of the catheter is positioned during operation, non-uniform rotation can be difficult to prevent.

In at least some embodiments, a motor capable of rotating the imaging core may be disposed on an imaging core positioned in a distal portion of the catheter. Typically, the imaging core has a longitudinal length that is substantially less than a longitudinal length of the catheter. The imaging core also includes one or more transducers. In at least some embodiments, disposing the motor in the imaging core may reduce, or even eliminate non-uniform rotation caused by one or more off-axis forces (e.g., blood vessel walls pressing against portions of the catheter). In at least some embodiments, the motor includes a rotor formed from a permanent magnet. In at least some embodiments, the catheter has an outer diameter that is no greater than one millimeter.

It may be the case that the distal end of the catheter 102 is disposed in patient vasculature without having any information regarding the precise location or orientation of the one or more transducers. In at least some embodiments, a sensing device may be disposed in the imaging core for sensing the location or orientation of the one or more transducers. In at least some embodiments, the sensing device includes one or more magnetic sensors. In some embodiments, the sensing device includes a plurality of magnetic sensors located external to the patient. In other embodiments, one or more sensors are positioned within the patient, and a plurality of sensors are positioned external to the patient.

Additionally or alternatively, in at least some embodiments, the sensing device measures the amplitude or orientation of the rotating magnet magnetization vector produced by the motor. In at least some embodiments, data from the magnetic sensing device may be input to a drive circuit to provide controlled and uniform rotation of the imaging core (e.g., through a feedback loop). In at least some embodiments, data from the sensing device may also be used to make corrections to data collected during non-uniform rotation of the imaging core.

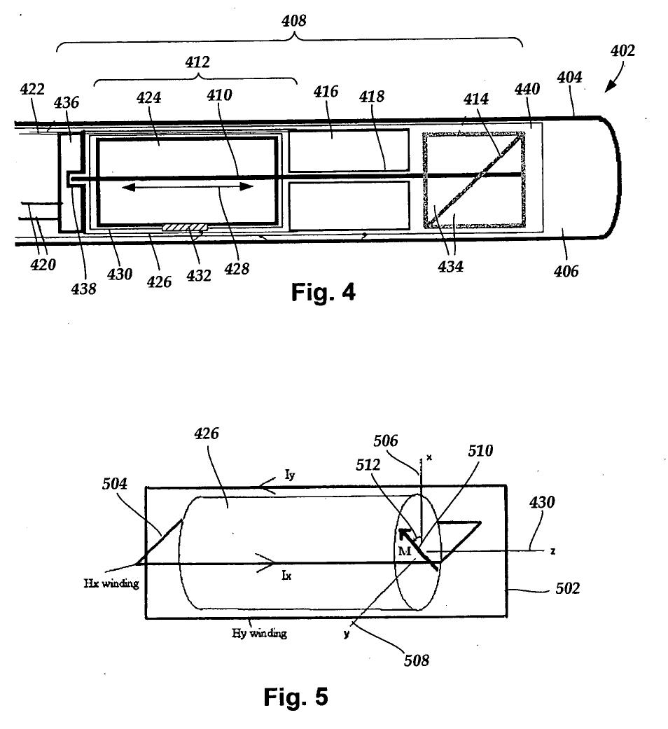

FIG. 4 is a schematic longitudinal cross-sectional view of one embodiment of a distal end of a catheter 402. The catheter 402 includes a sheath 404 and a lumen 406. A rotatable imaging core 408 is disposed in the lumen 406 at the distal end of the catheter 402. In at least some embodiments, the imaging core 408 is surrounded by an sonolucent fluid. In at least some embodiments, the fluid is airless. In at least some embodiments, the fluid has impedance that matches impedance of patient tissue at a target imaging site within the patient.

The imaging core 408 includes a rotatable driveshaft 410 with a motor 412 and a mirror 414 coupled to the driveshaft 410 and configured and arranged to rotate with the driveshaft 410. The imaging core 408 also includes one or more transducers 416 defining an aperture 418 extending along a longitudinal axis of the one or more transducers 416. In at least some embodiments, the one or more transducers 416 are positioned between the motor 412 and the mirror 414. In at least some embodiments, the one or more transducers 416 are configured and arranged to remain stationary while the driveshaft 410 rotates. In at least some embodiments, the driveshaft 410 extends through the aperture 418 defined in the one or more transducers 416. In at least some embodiments, the aperture 418 is formed from a material, or includes a coating, or both, such as polytetrafluoroethylene coated polyimide tubing, that reduces drag between the rotatable driveshaft 410 and the stationary (relative to the driveshaft 410) aperture 418 of the one or more transducers 416.

One or more motor conductors 420 electrically couple the motor 412 to the control module (104 in FIG. 1). In at least some embodiments, the one or more of the motor conductors 420 may extend along at least a portion of the longitudinal length of the catheter 402 as shielded electrical cables, such as a coaxial cable, or a twisted pair cable, or the like. One or more transducer conductors 422 electrically couple the one or more transducers 416 to the control module (104 in FIG. 1). In at least some embodiments, the one or more of the catheter conductors 422 may extend along at least a portion of the longitudinal length of the catheter 402 as shielded electrical cables, such as a coaxial cable, or a twisted pair cable, or the like.

In at least some embodiments, the outer diameter of the catheter 402 is no greater than 0.042 inches (0.11 cm). In at least some embodiments, the outer diameter of the catheter 402 is no greater than 0.040 inches (0.11 cm). In at least some embodiments, the outer diameter of the catheter 402 is no greater than 0.038 inches (0.10 cm). In at least some embodiments, the outer diameter of the catheter 402 is no greater than 0.036 inches (0.09 cm). In at least some embodiments, the outer diameter of the catheter 402 is no greater than 0.034 inches (0.09 cm). In at least some embodiments, the outer diameter of the catheter 402 is sized to accommodate intracardiac echocardiography systems.

The motor 412 includes a rotor 424 and a stator 426. In at least some embodiments, the rotor 424 is a permanent magnet with a longitudinal axis 428 (shown in FIG. 4 as a two-headed arrow) that is parallel to a longitudinal axis of the driveshaft 410. The magnet 424 may be formed from many different magnetic materials suitable for implantation including, for example, neodymium-iron-boron, or the like. One example of a suitable neodymium-iron-boron magnet is available through Hitachi Metals America Ltd, San Jose, Calif.

In at least some embodiments, the outer diameter of the magnet 424 is no greater than 0.025 inches (0.06 cm). In at least some embodiments, the outer diameter of the magnet 424 is no greater than 0.022 inches (0.06 cm). In at least some embodiments, the outer diameter of the magnet 424 is no greater than 0.019 inches (0.05 cm). In at least some embodiments, the longitudinal length of the magnet 424 is no greater than 0.13 inches (0.33 cm). In at least some embodiments, the longitudinal length of the magnet 424 is no greater than 0.12 inches (0.30 cm). In at least some embodiments, the longitudinal length of the magnet 424 is no greater than 0.11 inches (0.28 cm).

In at least some embodiments, the magnet 424 is cylindrical. In at least some embodiments, the magnet 424 has a magnetization M of no less than 1.4 T. In at least some embodiments, the magnet 424 has a magnetization M of no less than 1.5 T. In at least some embodiments, the magnet 424 has a magnetization M of no less than 1.6 T. In at least some embodiments, the magnet 424 has a magnetization vector that is perpendicular to the longitudinal axis of the magnet 424.

In at least some embodiments, the magnet 424 is disposed in a housing 430. In at least some embodiments, the housing 430 is formed, at least in part, from a conductive material (e.g., carbon fiber and the like). In at least some embodiments, the rotation of the magnet 424 produces eddy currents which may increase as the angular velocity of the magnet increases. Once a critical angular velocity is met or exceeded, the eddy currents may cause the magnet to levitate. In a preferred embodiment, the conductive material of the housing 430 has conductivity high enough to levitate the magnet 424 to a position equidistant from opposing sides of the housing 430, yet low enough to not shield the magnet 424 from a magnetic field produced by the stator 426.

In at least some embodiments, a space between the magnet 424 and the housing 430 is filled with a magnetic fluid suspension (“ferrofluid”) (e.g., a suspension of magnetic nano-particles, such as available from the Ferrotec Corp., Santa Clara, Calif.). The ferrofluid is attracted to the magnet 424 and remains positioned at an outer surface of the magnet 424 as the magnet 424 rotates. The fluid shears near the walls of non-rotating surfaces such that the rotating magnet 424 does not physically contact these non-rotating surfaces. In other words, if enough of the surface area of the magnet 424 is accessible by the ferrofluid, the ferrofluid may cause the magnet 424 to float, thereby potentially reducing friction between the magnet 424 and other contacting surfaces which may not rotate with the magnet 424 during operation. In at least some embodiments, the resulting viscous drag torque on the magnet 424 increases in proportion to the rotation frequency of the magnet 424, and may be reduced relative to a non-lubricated design.

The magnet 424 is coupled to the driveshaft 410 and is configured and arranged to rotate the driveshaft 410 during operation. In at least some embodiments, the magnet 424 is rigidly coupled to the driveshaft 410. In at least some embodiments, the magnet 424 is coupled to the driveshaft 410 by an adhesive.

In at least some embodiments, the stator 426 includes at least two perpendicularly-oriented magnetic field windings (502 and 504 in FIG. 5) which provide a rotating magnetic field to produce torque causing rotation of the magnet 424. The stator 426 is provided with power from the control module (104 in FIG. 1) via the one or more motor conductors 420.

In at least some embodiments, a sensing device 432 is disposed on the imaging core 408. In at least some embodiments, the sensing device 432 is coupled to the housing 432. In at least some embodiments, the sensing device 432 is configured and arranged to measure the amplitude of the magnetic field in a particular direction. In at least some embodiments, the sensing device 432 uses at least some of the measured information to sense the angular position of the magnet 424. In at least some embodiments, at least some of the measured information obtained by the sensing device 432 is used to control the current provided to the stator 426 by the one or more motor conductors 420. In at least some embodiments, the sensing device 432 can be used to sense the angular position of the mirror 414.

In at least some embodiments, acoustic signals may be emitted from the one or more transducers 416 towards the rotating mirror 414 and redirected to an angle that is not parallel to the longitudinal axis 428 of the magnet 424. In at least some embodiments, acoustic signals may be redirected to a plurality of angles that are within a 120 degree range with respect to the longitudinal axis 428 of the magnet 424. In at least some embodiments, acoustic signals may be redirected to a plurality of angles that are within a 90 degree range with respect to the longitudinal axis 428 of the magnet 424. In at least some embodiments, acoustic signals may be redirected to a plurality of angles that are within a 120 degree range with respect to the longitudinal axis 428 of the magnet 424 such that the plurality of angles are centered on an angle that is perpendicular to the longitudinal axis 428 of the magnet 424. In at least some embodiments, acoustic signals may be redirected to a single angle that is perpendicular to the longitudinal axis 428 of the magnet 424. In at least some embodiments, acoustic signals may be redirected to a single angle that is not perpendicular to the longitudinal axis 428 of the magnet 424.

In at least some embodiments, the mirror 414 is sandwiched between sonolucent material 434. In at least some embodiments, the sonolucent material is solid or semi-solid. In at least some embodiments, the sonolucent material 434 has impedance that matches the impedance of the sonolucent fluid surrounding the imaging core 408. In at least some embodiments, the sonolucent material 434 is disposed over the mirror 414 such that the mirror 414 and sonolucent material 434 form a structure with an even weight distribution around the driveshaft 410. In at least some embodiments, the sonolucent material 434 is disposed over the mirror 414 such that the mirror 414 and sonolucent material 434 form a cylindrically-shaped structure.

In at least some embodiments, the mirror 414 includes a reflective surface that is planar. In at least some embodiments, the mirror 414 includes a reflective surface that is non-planar. In at least some embodiments, the reflective surface of the mirror 414 is concave. It may be an advantage to employ a concaved reflective surface to improve focusing, thereby improving lateral resolution of acoustic pulses emitted from the catheter 402. In at least some embodiments, the reflective surface of the mirror 414 is convex. In at least some embodiments, the shape of the reflective surface of the mirror 414 is adjustable. It may be an advantage to have an adjustable reflective surface to adjust the focus or depth of field for imaging tissues at variable distances from the mirror 414.

In at least some embodiments, the imaging core 108 includes a proximal end cap 436. In at least some embodiments, the proximal end cap 436 provides structure to the proximal portion of the imaging core 108. In at least some embodiments, the proximal end cap 436 is rigid enough to withstand lateral forces (i.e., off-axis forces) typically encountered during normal operation within patient vasculature such that the operation of the motor 412 is not interrupted. In at least some embodiments, a proximal end of the driveshaft 410 contacts the proximal end cap 436. In at least some embodiments, the proximal end cap 436 defines a drag-reducing element 438 for reducing drag caused by the rotating driveshaft 410 contacting the proximal end cap 436. The drag-reducing element 438 can be any suitable device for reducing drag including, for example, one or more bushings, one or more bearings, or the like or combinations thereof. In at least some embodiments, the drag-reducing element 438 facilitates uniformity of rotation of the driveshaft 410.

In at least some embodiments, the catheter 402 includes an inner sheath 440 surrounding the imaging core 408. In at least some embodiments, the inner sheath 440 physically contacts at least one of the motor 412 or the one or more transducers 416, but does not physically contact the rotating mirror 414 during normal operation of the imaging core 408. In at least some embodiments, the inner sheath 440 is rigid. In at least some embodiments, the inner sheath 440 is rigid enough to withstand lateral forces (i.e., off-axis forces) typically encountered during normal operation within patient vasculature such that the mirror 414 does not contact the inner sheath 440. In at least some embodiments, the inner sheath 440 is filled with a sonolucent fluid. In at least some embodiments, the sonolucent fluid has impedance that matches the impedance of the sonolucent fluid within the lumen 404 of the catheter 402.

In at least some embodiments, the motor 412 provides enough torque to rotate the one or more transducers 416 at a frequency of at least 15 Hz. In at least some embodiments, the motor 412 provides enough torque to rotate the one or more transducers 416 at a frequency of at least 20 Hz. In at least some embodiments, the motor 412 provides enough torque to rotate the one or more transducers 416 at a frequency of at least 25 Hz. In at least some embodiments, the motor 412 provides enough torque to rotate the one or more transducers 416 at a frequency of at least 30 Hz. In at least some embodiments, the motor 412 provides enough torque to rotate the one or more transducers 416 at a frequency of at least 35 Hz. In at least some embodiments, the motor 412 provides enough torque to rotate the one or more transducers 416 at a frequency of at least 40 Hz.

In a preferred embodiment, the torque is about the longitudinal axis 428 of the magnet 424 so that the magnet 424 rotates. In order for the torque of the magnet 424 to be about the longitudinal axis 428 of the magnet 424, the magnetic field of the magnetic field windings (i.e., coils of the stator 426) lies in the plane perpendicular to the longitudinal axis 428 of the magnet 424, with the field vector rotating about the longitudinal axis 428 of the magnet 424.

As discussed above, the stator 426 provides a rotating magnetic field to produce a torque on the magnet 424. The stator 426 may comprise two perpendicularly-oriented magnetic field windings (“windings”) that wrap around the magnet 424 as one or more turns to form a rotating magnetic field. FIG. 5 is a schematic perspective view of one embodiment of the rotating magnet 424 and windings, represented as orthogonal rectangular boxes 502 and 504. Although the windings 502 and 504 are shown as two orthogonal rectangles, it will be understood that the each of the windings 502 and 504 may represent multiple turns of wire which may be spread out to minimize an increase in the outer diameter of the catheter (402 in FIG. 4). When the windings 502 and 504 are spread out, a band of current may be generated instead of the lines of current shown in FIG. 5.

In at least some embodiments, the diameter of the wire used to form the windings 502 and 504 is no greater than 0.004 inches (0.010 cm). In at least some embodiments, the diameter of the wire is no greater than 0.003 inches (0.008 cm). In at least some embodiments, the diameter of the wire is no greater than 0.002 inches (0.005 cm).

In order for the magnet 424 to rotate about the longitudinal axis 428, the torque must be about the longitudinal axis 428. Therefore, the magnetic field generated by the windings 502 and 504 must lie in a plane perpendicular to the longitudinal axis 428 with a magnetic field vector H for the windings 502 and 504 rotating about the longitudinal (z) axis 430 to torque and rotate the magnet 424. FIG. 5 also shows an x-axis 506 and a y-axis 508 that are orthogonal to each other and to the longitudinal axis 428. As shown in FIG. 5, the magnetization vector M 510 of the magnet 402 is in an x-y plane that is perpendicular to the longitudinal axis 428.

The winding 502 produces a magnetic field at the center of the winding 502 that is parallel to the y-axis 508. The winding 504 produces a magnetic field at the center of the winding 504 that is parallel to the x-axis 506. The combined magnetic field vector H for the windings 502 and 504 is given by:

H=Hₓ x′+H_{y} y′.

where x′ and y′ are unit vectors in the x and y directions, respectively. The magnetization vector M rotates through the angle 512, which is equal to the angular velocity of the magnet 424 times the elapsed time for uniform rotation. Thus, the magnetization vector M is given by:

M=M (cos(ωt) x′+sin(ωt) y′).

The magnetic moment vector m is given by:

m=MV;

where M=magnetization vector of the magnet 424 in Tesla; and V=the magnet 424 volume in m³.

The torque τ exerted on the magnet 424 is given by:

τ=m×H;

where τ=the torque vector in N-m; m=the magnetic moment vector in Tesla-m³; H=the magnetic field vector of the windings 502 and 504 in amp/m; and x=the vector cross product.

The vector cross product can be evaluated:

τ=MV (H_{y} cos(ωt)−Hₓ sin(ωt)) z′.

The vector cross product verifies that the torque produced by the windings 502 and 504 on the magnetic moment vector m is indeed about the longitudinal axis 428. Moreover, the torque will be uniform and independent of time if the magnetic fields generated by the windings 502 and 504 are given by:

Hₓ=−H sin(ωt);

H_{y}=H cos(ωt);

thereby yielding a torque τ given by:

τ=MVHz′.

The torque is uniform because the magnetic field is uniformly rotating, since H²=Hₓ²+H_{y}² is independent of time, and the Hₓ and H_{y} components describe clockwise rotation of the winding magnetic field vector H about the z′ axis. The resulting uniform torque on a symmetric magnet having the magnetization vector M in the x-y plane is an inherent expression of a rotating field electric motor.

Thus, the orthogonal fields produce a magnetic field that uniformly rotates about the longitudinal axis 428 at angular speed ω. Under operational conditions, the magnetization vector M of the magnet 424 will follow the winding magnetic field vector H of the windings 502 and 504 with a slip angle that is determined by a system drag torque. When the angular speed c is increased, the drag torque (and the slip angle) increases until the magnet 424 can no longer rotate fast enough to keep up with the magnetic field.

A changing slip angle may potentially lead to non-uniform rotation. In at least some embodiments, the sensing device 432 facilitates maintaining uniform rotation of the magnet 424 by maintaining a uniformly rotating magnetic field. In at least some embodiments, the sensing device 432 controls the currents that produce Hₓ and H_{y} by feedback from measured values for Mₓ and M_{y} components. The relationship between Hₓ and H_{y} and Mₓ and M_{y} is given by:

Hₓ ∝ Iₓ ∝ −M_{y}; and

H_{y} ∝ I_{y} ∝ Mₓ;

where Iₓ=the current in amps producing the magnetic field component Hₓ; and I_{y}=the current in amps producing the magnetic field component H_{y}.

In at least some embodiments, the sensing device 432 may be implemented in digital form. In at least some embodiments, digitally processed data output from the sensing device 432 is used to compute the currents at each point in time to maintain uniform rotation. In at least some embodiments, the digital sensing device 432 may measure more than one component of the magnetic field of the magnet 424 at a given point to fully determine the currents for a given rotational direction.

In at least some other embodiments, the sensing device 432 may be implemented in analog form. In at least some embodiments, the analog sensing device 432 includes two magnetic sensors placed 90 degrees apart on the housing (430 in FIG. 4) or elsewhere on the imaging core (408 in FIG. 4). Generally, the magnetic field generated by the magnet 424 is substantially larger than the magnetic field generated by the windings 502 and 504. Thus, the sensors of the sensing device 432 measure the perpendicular components of the magnetization vector M in the x-y plane, relative to the axes passing from the center of the magnet 424 to the sensors. The measured signals can be amplified and fed back to the currents in the windings 502 and 504. If, as shown in the previous equations, the x current is inverted, the magnet 424 rotates clockwise. If the y current is inverted, the magnet 424 rotates counterclockwise.

In at least some embodiments, the sensing device 432 includes at least some magnetic sensors located external to the patient. For example, two tri-axial magnetic sensors, including six individual sensors, may measure the x, y, and z components of a rotating magnetic field of the magnet 424 at two locations external to the patient. In at least some embodiments, magnetic field sensing of the rotating magnet 424 is facilitated by sensing only magnetic fields that rotate in phase with the magnet winding drive currents. Data from the external sensors may be inverted to find the x, y, and z coordinates of the rotating magnet (and IVUS transducer), and the spatial orientation of the magnet 424. This data can be used to form a three dimensional image of surrounding tissue (e.g., bends in an artery) during pull back imaging.

In at least some embodiments, one or more sensors may be positioned in proximity to the rotating magnet 424 and implantable into the patient, while a plurality of sensors remain external to the patient. The implantable sensor may identify the angular orientation of the rotating magnet 424, and this data may be used to accept only data from the external sensors that have the proper frequency and proper phase angle of the rotating magnet while rejecting data obtained from external sensors with an improper frequency and phase angle, thereby further increasing the signal-to-noise ratio in the external sensor data.

The amount of magnetic torque that may be generated by the motor 412 may be limited by the amount of current that may be passed through the windings 502 and 504 without generating excessive heat in the catheter (402 in FIG. 4). Heat is generated in the windings 502 and 504 by Joule heating at a rate given by:

P=I²R;

where P=the power dissipated as heat in watts; R=the resistance of the windings 502 and 504; and I=the amplitude of the current in amps.

The value for P is divided by two because sinusoidal current is employed. However the value for P is also multiplied by two because there are two windings 502 and 504. In at least one experiment, it has been estimated that up to 300 mW of heat is readily dissipated in blood or tissue without perceptibly increasing the temperature of the motor (412 in FIG. 4). In at least one experiment, it has been estimated that heat dissipation increases to several watts when blood is flowing.

The magnetic field H of the windings 502 and 504 having N turns and inputting current I may be computed. The result follows from the formula for the magnetic field generated by a current-carrying line segment. Typically, the lengths of the long ends of the rectangular-shaped windings 502 and 504 parallel with the longitudinal axis 428 are substantially greater than the lengths of the short ends of the windings 502 and 504. Accordingly, the short ends may not significantly contribute to the magnetic torque. The magnetic field H of the windings 502 and 504 having N turns and inputting current I is given by:

H=2NI/(πD{square root over ((+(D/L)²))});

where N=the number of turns of the windings 502 and 504; D=the winding width in meters (typically the outer diameter of the housing (430 in FIG. 4); and L=the length of the windings 502 and 504 in meters. NI can be analyzed in terms of the power dissipated in the windings 502 and 504. Although theoretical optimization of all parameters is possible, safety limits may be incorporated into design implementation.

In one exemplary embodiment, rectangular windings 502 and 504 have 8 turns of silver wire with a 2.7 inches (6.86 cm) length, a 0.002 inch (0.005 cm) diameter, and a resistance of 0.5 Ohms. A magnet 424 has a cylindrical shape with an outer diameter of 0.022 inches (0.056 cm), an inner diameter of 0.009 inches (0.022 cm), and a longitudinal length of 0.132 inches (0.34 cm). The magnetization M=1.4 for the magnet 424 having the above-mentioned dimensions formed from neodymium-iron-boron. The maximum power P is equal to 0.3 watts, the maximum current amplitude is 0.77 amps, and the quantity NI is 6.2 amps. Using the above-mentioned values, the torque on the magnet 424 is given by:

τ=2MV(NI)/(πD{square root over ((1+(D/L)²))}).

Inserting the above-mentioned values gives a torque of 4 μN-m=0.4 μm-mm, which is approximately four times larger than an estimated maximum frictional drag on the magnet 424. The corresponding force is about 0.1 gram, or about 30 times the weight of the magnet 424. Although torque may be increased by increasing the magnet radius, it is desirable that the catheter (402 in FIG. 4) be small enough to be disposed in a wide variety of patient vasculature. Additional considerations for insertion of the catheter into patient vasculature may be considered including, for example, the length of the imaging core (408 in FIG. 4) (because the relative stiffness of the imaging core (408 in FIG. 4) may affect maneuverability of the catheter), heat generation, the resistivity of metals at room temperature, and the strength of the materials used to form the magnet 424.

In at least some embodiments, up to six amps of current may be utilized by the motor. Thus, in a preferred embodiment, the components of the catheter and imaging core are capable of withstanding up to six amps of current without heating. Low power electronic components are currently available to source six amps of current at low voltage. Additionally, previous studies have shown that flexible stranded leads with an equivalent diameter of approximately 0.015 inches (0.04 cm) can withstand up to six amps of current, while also being capable to fitting through a catheter with a one-millimeter outer diameter.

It may be difficult to form the windings 502 and 504. For example, it may be difficult to wind a wire of 0.002 inch (0.005 cm) diameter around a cylindrical surface of a housing (432 in FIG. 4). In at least some embodiments, the windings 502 and 504 are deposited onto a thin film (e.g., a polyimide film, or the like), which is then disposed onto the housing (432 in FIG. 4). For example, one or more types of metals (e.g., copper, silver, gold, or other metals or metal alloys) are deposited onto the thin film, and the thin film is disposed onto the housing (e.g., using one or more adhesives or other types of suitable coupling methods). In alternate embodiments, the housing (430 in FIG. 4) is formed from a ceramic cylinder or extruded polyimide tube, or other material that is suitable for deposition of metal strip lines. A three-dimensional lithography process may be used to deposit and define the windings 502 and 504 on the cylinder. For example, a metal film may be deposited uniformly on an outer surface of the cylinder and a laser may be used to remove undesired metal film from the outer surface of the cylinder, thereby defining the windings 502 and 504.

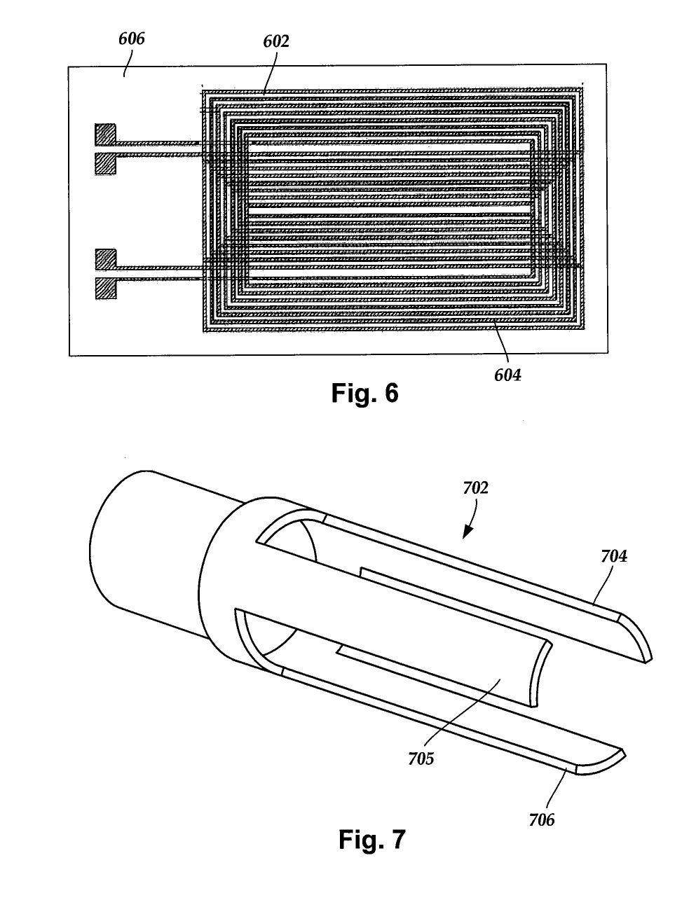

FIG. 6 is a schematic top view of one embodiment of the windings 602 and 604 disposed on a thin film 606. In at least some embodiments, the windings 602 and 604 are disposed on both sides of the thin film 606. In at least some embodiments, the winding 602 is disposed on a first side of the thin film 606 and the winding 604 is disposed on a second side of the thin film 606. In preferred embodiments, the windings 602 and 604 are disposed on the thin film 606 such that when the thin film 606 is disposed around the magnet 424 (or the housing 430), the windings 602 and 604 are offset from one another by 90 degrees.

In preferred embodiments, the stator 426 is formed from rigid or semi-rigid materials using multiple-phase winding geometries. It will be understood that there are many different multiple-phase winding geometries and current configurations that may be employed to form a rotating magnetic field. For example, the stator 426 may include, for example, a two-phase winding, a three-phase winding, a four-phase winding, a five-phase winding, or more multiple-phase winding geometries. It will be understood that a motor may include many other multiple-phase winding geometries. In a two-phase winding geometry, for example, the currents in the two windings are out of phase by 90°. For a three-phase winding, there are three lines of sinusoidal current that are out of phase by zero, 120°, and 240°, with the three current lines also spaced by 120°, resulting in a uniformly rotating magnetic field that can drive a cylindrical rotor magnet magnetized perpendicular to the current lines.

FIG. 7 is a schematic perspective view of one embodiment of a three-phase winding geometry 702 configured and arranged for forming a rotating magnetic field around a magnet (see e.g., 424 in FIG. 4). The three-phase winding 702 includes three arms 704-706 onto which windings can be disposed. In at least some embodiments, multiple windings may utilize a single cylindrical surface of the magnet (424 of FIG. 4) with no cross-overs. Such a winding may occupy a minimal volume in an imaging core. Although other geometries may also form a rotating magnetic field, the three-phase geometry 702 may have the advantages of allowing for a more compact motor construction than other geometries.

An exceptional property of a three-phase winding geometry 702 is that only two of the three windings disposed on the arms 704-706 need to be driven, while the third winding is a common return that mathematically is equal to the third phase of current. This can be verified by noting that:

Sin (ωt)+Sin(ωt+120°)=−Sin(ωt+240°)

For a three-phase winding geometry 702, current is driven into two lines with the zero and 120° phase shift of the two terms on the left side of this identity. The sum of the two terms returns on the common line with exactly the correct 240° phase shift on the right side of this equation needed to create the rotating magnetic field. It will be understood that the minus sign indicates that the return current is in the opposite direction of driven current.

In at least some embodiments, the arms 704-706 may be supported by a substrate to increase mechanical stability. In at least some embodiments, the arms 704-706 are constructed from a solid metal tube (e.g., a hypotube, or the like), leaving most of the metal in tact, and removing only metal needed to prevent electrical shorting between the lines 704-706. For example, in at least some embodiments, the arms 704-706 are formed from a cylindrical material with a plurality of slits defined along at least a portion of a longitudinal length of the arms 704-706, at least some of the slits separating adjacent windings.

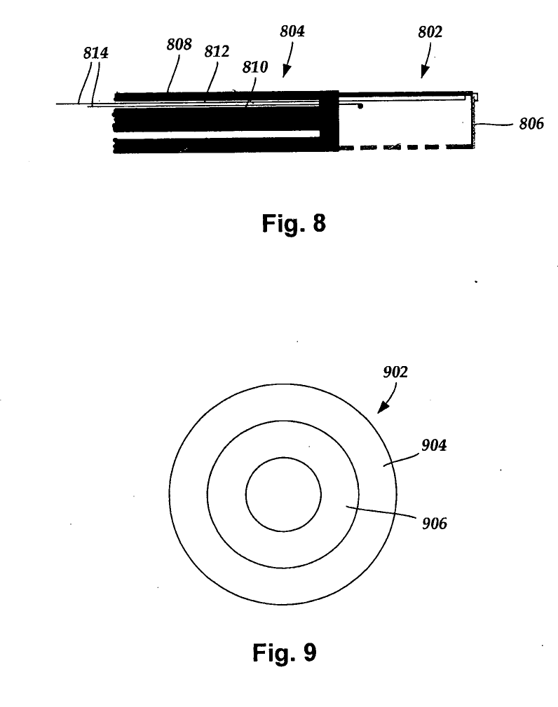

FIG. 8 is a schematic side view of one embodiment of a portion of a transducer 802 coupled to a portion of a stator 804. The transducer 802 includes a front face 806 from which acoustic signals can be emitted. The stator 804 includes windings disposed on arms, such as arms 808 and 810 separated from one another by longitudinal slits, such as slit 812 separating arm 808 from arm 810. Transducer conductors 814 electrically couple the transducer 802 to the control module (104 in FIG. 1). In at least some embodiments, the transducer conductors 814 extend along at least a portion of one or more of the slits (such as slit 812) extending along the longitudinal length of the stator 804. It may be an advantage to extend the transducer conductors 814 along one or more of the slits of the stator 804 to potentially reduce the diameter of the imaging core (see e.g., 408 of FIG. 4). In at least some embodiments, at least a portion of the stator 804 extends over at least a portion of the transducer 802. In at least some embodiments, the portion of the stator 804 extending over the portion of the transducer 802 extends such that radial return currents occur far enough distal to the magnet (424 in FIG. 4) to produce only negligible torque on the magnet (424 in FIG. 4).

In at least some embodiments, the one or more transducers include a plurality of annuli. In at least some embodiments, at least one of the annuli resonates at a frequency that is different from at least one of the remaining annuli. FIG. 9 is a schematic transverse cross-sectional view of one embodiment of a transducer 902 with a plurality of annuli, such as annulus 904 and annulus 906. In at least some embodiments, the annulus 904 resonates at a different frequency than the annulus 906.

As shown above, the torque on the motor (412 in FIG. 4) is given by:

τ=2MV(NI)/(πD{square root over ((1+(D/L)²))});

wherein the only dependence of torque on the windings is through the product NI. For example, the same result is obtained regardless of whether 0.77 amps flow through windings with 8 turns, or 6.2 amps flow through windings with 1 turn. Heat generation will be the same as long as the total cross-sectional area of the windings is the same. For example, one line two mills high and sixteen mills wide heats equivalent to eight lines two mills high and two mills wide. Accordingly, in at least some embodiments, each winding includes a single turn.

It will be understood that the rotating shaft of the motor may be used for other applications, including pumping blood, ablating tissue, providing propulsion to move or steer the distal tip, and the like or combinations thereof.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A catheter assembly for an intravascular ultrasound system, the catheter assembly comprising:
a catheter having a longitudinal length, a distal end, and a proximal end, the catheter comprising a lumen extending along the longitudinal length of the catheter from the proximal end to the distal end;
an imaging core with a longitudinal length that is substantially less than the longitudinal length of the catheter, the imaging core configured and arranged for inserting into the lumen and disposing at the distal end of the catheter, the imaging core comprising
a rotatable driveshaft having a distal end and a proximal end,
a rotatable mirror disposed at the distal end of the driveshaft,
a motor coupled to the driveshaft, the motor comprising a rotatable magnet and at least two magnetic field windings disposed around at least a portion of the magnet, wherein the magnetic field windings are disposed on a rigid slotted material, and
at least one fixed transducer positioned between the motor and the rotatable mirror, the at least one transducer having an aperture defined along a longitudinal axis of the at least one transducer, the aperture configured and arranged to allow passage of the driveshaft through the at least one transducer to the rotatable mirror, the at least one transducer configured and arranged for transforming applied electrical signals to acoustic signals and also for transforming received echo signals to electrical signals;
at least one transducer conductor electrically coupled to the at least one transducer and in electrical communication with the proximal end of the catheter; and
at least one motor conductor electrically coupled to the magnetic field windings and in electrical communication with the proximal end of the catheter.

2. The catheter assembly of claim 1, wherein the imaging core further comprises a proximal hub disposed at the proximal end of the driveshaft, the proximal hub providing structural support proximal to the motor.

3. The catheter assembly of claim 1, wherein the mirror is tilted at an angle such that when an acoustic beam is emitted from the at least one transducer to the mirror, the acoustic beam is redirected in a direction that is not parallel the longitudinal axis of the magnet.

4. The catheter assembly of claim 1, wherein the at least one transducer conductor extends along at least a portion of at least one of the slots defined in the magnetic field windings.

5. The catheter assembly of claim 1, wherein the imaging core further comprises a sensing device, the sensing device configured and arranged for sensing the angular position of the magnet.

6. The catheter assembly of claim 5, wherein the sensing device is configured and arranged to control an amount of current applied to the magnetic field windings using the received angular position of the magnet.

7. The catheter assembly of claim 1, wherein the catheter has a transverse outer diameter that is not greater than one millimeter.

8. The catheter assembly of claim 1, wherein the magnet is disposed in a housing.

9. The catheter assembly of claim 8, wherein the housing is formed from a conductive material with conductivity high enough to levitate the magnet when the magnet rotates at an operational angular velocity.

10. The catheter assembly of claim 8, wherein the magnetic field windings are disposed on a thin film disposed over the housing.

11. The catheter assembly of claim 1, wherein the at least one fixed transducer is mounted to the magnetic field windings.

12. The catheter assembly of claim 1, wherein the magnetic field windings each comprise a single turn of a conductive material.

13. The catheter assembly of claim 1, wherein the mirror comprises a non-planar reflective surface.

14. The catheter assembly of claim 1, wherein the at least one transducer comprises a plurality of annuli, at least one annulus configured and arranged to resonate at a frequency that is different from at least one other annulus.

15. The catheter assembly of claim 1, wherein the mirror is disposed within sonolucent material having impedance that matches patient tissue impedance in proximity to the distal end of the catheter, wherein the sonolucent material is positioned to have an even weight distribution around the driveshaft.

16. The catheter assembly of claim 15, wherein the mirror is disposed within the sonolucent material such that the mirror and the sonolucent material form a cylindrically-shaped structure.

17. The catheter assembly of claim 1, further comprising an inner sheath disposed over the imaging core within the lumen of the catheter, the inner sheath filled with a sonolucent fluid having impedance that matches patient tissue impedance in proximity to the distal end of the catheter.

18. The catheter assembly of claim 1, wherein the catheter is filled with a sonolucent fluid having impedance that matches patient tissue impedance in proximity to the distal end of the catheter.

19. An intravascular ultrasound imaging system comprising:
the catheter assembly of claim 1; and
a control module coupled to the imaging core, the control module comprising
a pulse generator configured and arranged for providing electric signals to the at least one transducer, the pulse generator electrically coupled to the at least one transducer via the at least one transducer conductor, and
a processor configured and arranged for processing received electrical signals from the at least one transducer to form at least one image, the processor electrically coupled to the at least one transducer via the at least one transducer conductor.

20. A method for imaging a patient using an intravascular ultrasound imaging system, the method comprising:
inserting a catheter into patient vasculature, the catheter comprising an imaging core disposed in a distal portion of a lumen defined in the catheter, the imaging core electrically coupled to a control module by at least one conductor, the imaging core having a longitudinal axis and comprising at least one fixed transducer, a tilted mirror, and a magnet that rotates by application of a current from the control module to at least two magnetic field windings wrapped around at least a portion of the magnet, wherein the transducer emits acoustic signals directed at the tilted mirror, and wherein the rotation of the magnet causes rotation of the mirror;
positioning the imaging core in a region to be imaged;
transmitting at least one electrical signal from the control module to the at least one transducer;
transmitting at least one electrical signal from the control module to the at least two magnetic field windings, the at least one electrical signal causing the magnet and the mirror to rotate;
transmitting at least one acoustic signal from the at least one transducer to patient tissue via reflection from the rotating mirror;
receiving at least one echo signal from a tissue-boundary between adjacent imaged patient tissue by the imaging core; and
transmitting at least one transformed echo signal from the at least one transducer to the control module for processing.
